(19) United States
(12) Patent Application Publication
(10) Pub. No.: US 2019/0237191 A1
(43) Pub. Date: Aug. 1, 2019

(54) A METHOD AND SYSTEM FOR GENERATING AN ASSESSMENT OF A TREATMENT RECOMMENDATION FOR A PATIENT

(71) Applicant: KONINKLIJKE PHILIPS N.V., EINDHOVEN (NL)

(72) Inventors: Thomas Netsch, Hamburg (DE); Nicole Schadewaldt, Norderstedt (DE); Heinrich Schulz, Hamburg (DE)

(21) Appl. No.: 16/339,133

(22) PCT Filed: Oct. 4, 2017
(86) PCT No.: PCT/EP2017/075166

§ 371 Date: Apr. 3, 2019

(30) Foreign Application Priority Data
Oct. 6, 2016 (EP) 16192510.2

(51) Int. Cl. G16H 50/20; G16H 50/70

(52) U.S. Cl. CPC G16H 50/20; G16H 50/70

(57) ABSTRACT

The invention provides a method and system for assessing a treatment recommendation (TR) for a patient. It involves analysis of the treatments provided to previous patients (pr) with the same general medical condition by a plurality of different treatment specialists. A measure of conformity (C) of the treatment recommendation to the medical condition (MC) of the patient is provided, based on the range of treatments received by previous patients having that medical condition, for example based on a relative value between the number of previous patients (npr) that received the recommended treatment and the number that received all possible treatments.

### FIELD OF THE INVENTION

This invention relates to the analysis of treatment recommendation for a patient, in particular to determine if it is consistent with other patients.

### BACKGROUND OF THE INVENTION

The invention is of particular interest for decision making relating to oncology treatments, although it may be applied more generally to treatments for other conditions.

The treatment of a patient in oncology is generally discussed and planned in a tumor board review meeting (or a similar multidisciplinary team meeting) trying to best match the patient medical condition with a required standard of care, via cancer treatment guidelines and also novel treatment options as appropriate.

A tumor board review facilitates multidisciplinary opinion sharing by including medical experts such as medical oncologists (who provide cancer treatment with drugs), surgeons (who provide cancer treatment with surgery), radiation oncologists (who provide cancer treatment with radiation) and pathologists.

It is known that an individual treatment decision taken is biased by the background and experience of the medical doctors as well as by the specialization of the particular hospital at which the decision is made. Therefore, many patients submit their case to tumor boards at multiple hospitals and cancer centers trying to find the best option among the different recommendations.

Unfortunately, multiple assessments do not necessarily consistently suggest the same or even a similar treatment option, but may vary significantly. At this point, the patient (or a medical expert) cannot evaluate how “objective” or how “good” a particular option is and the patient may finally choose the one which he or she just feels best about. Currently there is no means to quantitatively relate a specific recommendation to other patients with comparable medical conditions.

Of course, the patient may make a decision dependent on survival rates or other success criteria of certain cancer treatments, but these are often not specific enough to his or her specific condition and other patient-specific parameters. Furthermore, uncertainty remains whether the different options are complete or correctly chosen. As a result, the patient may ask yet another tumor board which will again provide no further measureable help in reaching a treatment decision.

There is therefore a need for a system and method which provides an objective indication of the suitability of a particular treatment recommendation.

### SUMMARY OF THE INVENTION

The invention is defined by the claims.

According to examples in accordance with an aspect of the invention, there is provided a method of generating an assessment of a treatment recommendation for a patient, comprising:

receiving a treatment recommendation for the patient;

receiving information about the medical condition of the patient;

receiving information including the number of previous patients who have received the recommended treatment and had a medical condition corresponding to that of the patient, wherein the number of previous patients include patients treated by a plurality of different treatment specialists;

receiving information including the number of previous patients who had a medical condition corresponding to that of the patient but received one or more alternative treatment options; and

providing a measure of conformity of the treatment recommendation to the medical condition of the patient based on the treatments received by previous patients having a medical condition corresponding to that of the patient.

This method is carried out after a treatment recommendation has been made. It then compares the recommended treatment with the treatments given to previous patients with the same medical condition, to provide a measure of conformity. This indicates how usual or unusual the recommended treatment is, having regard to the medical information. The treatments previously given are readily available as documented data. The conformity measure does not try to assess the quality of the decision made in reaching the recommendation, but provides a statistical measure which is of interest for the patient and for medical professional to judge how typical the recommendation is. It may be atypical because the patient has specific needs or wishes, or the fact that it is atypical may provide an indication that the decision making process should be looked at again.

The measure of conformity then provides additional data to accompany the treatment recommendation for assisting the interpretation of the treatment recommendation.

The method may comprise consulting health insurance information to obtain the previous patient treatment information and medical conditions. Such health insurance information is prepared in many countries in order to quantify the cost of treatment, and it includes coding of the patient condition as well as the treatment carried out. It thus provides the information needed for an independent benchmark concerning the treatment given to multiple patients by multiple medical institutions.

The measure of conformity may comprise the ratio of:

(i) the number of previous patients with matching information about their medical condition who had the treatment corresponding to the treatment recommendation, to

(ii) the total number of patients with matching information about their medical condition.

It thus finds the proportion of similar patients who had the same treatment. The more patient information that is coded into the “information about the medical condition”, the more the conformity measure will be tailored to the specific type of patient.

Thus, the information about the medical condition of the patient may further comprise patient-specific information relating to the medical condition. This goes beyond a simple classification of the type of condition, but includes other factors.

The patient-specific information for comprises one or more of:

patient condition (for example including the level of disease progression e.g. size and/or number and location of tumors, and other general medical information such as heart rate, blood pressure etc.);

patient family history; and

biomarker information for the patient.

The biomarker information for example relates to the presence of genetic markers which may indicate a predisposition to particular conditions.

The method may comprise generating a plurality of measures of conformity relating to different types of patient-specific information. Thus, different measures may be used.

For one or more of the different types of patient-specific information, the method may comprise generating a histogram which records the different treatments received by different previous patients for different values of the particular type of patient-specific information. Histograms provide a simple way to analyze the data, based on the classification (i.e. histogram column) in which the patient sits.

The medical condition for example comprises a cancerous tumor and the treatment recommendation may then comprise chemotherapy, surgery or radiotherapy.

The method may be implemented in software.

Examples in accordance with another aspect of the invention provide a system for generating an assessment of a treatment recommendation for a patient, comprising:

an input for receiving:
a treatment recommendation for the patient;
information about the medical condition of the patient;
information including the number of previous patients who have received the recommended treatment and had a medical condition corresponding to that of the patient, wherein the number of previous patients include patients treated by a plurality of different treatment specialists; and
information including the number of previous patients who had a medical condition corresponding to that of the patient but received one or more alternative treatment options; and

a processor, which is adapted to:
provide a measure of conformity of the treatment recommendation to the medical condition of the patient based on the treatments received by previous patients having a medical condition corresponding to that of the patient.

This system generates a measure of conformity to accompany a treatment recommendation. The processor may be adapted to consult health insurance information to obtain the previous patient treatment information and medical conditions.

The processor may determine, as the measure of conformity, the ratio of:

(i) the number of previous patients with matching information about their medical condition who had the treatment corresponding to the treatment recommendation, to

(ii) the total number of patients with matching information about their medical condition.

As explained above, the information about the medical condition of the patient may further comprise patient-specific information such as the patient condition, patient family history and biomarker information for the patient, and then a plurality of measures of conformity may then be generated.

### BRIEF DESCRIPTION OF THE DRAWINGS

Examples of the invention will now be described in detail with reference to the accompanying drawings, in which:

FIG. 1 shows a method of generating an assessment of a treatment recommendation for a patient;

FIG. 2 shows a system for generating an assessment of a treatment recommendation for a patient;

FIG. 3 shows how patient-specific medical data may be taken into account; and

FIG. 4 shows the general architecture of a computer which may be used to implement the method of the invention.

### DETAILED DESCRIPTION OF THE EMBODIMENTS

The invention provides a method and system for assessing a treatment recommendation for a patient. It involves analysis of the treatments provided to patients with the same general medical condition by a plurality of different treatment specialists. A measure of conformity of the treatment recommendation to the medical condition of the patient is provided, based on the range of treatments received by previous patients having that medical condition, for example based on a relative value between the number of previous patients that received the recommended treatment and the number that received all possible treatments.

In this way, there is assessment of the conformity of a recommended treatment option with the medical condition of an individual patient. A high conformity measure value means that many other patients receiving this treatment have similar medical conditions to the patient in question, for example a large number of tumor boards advised this treatment under the same conditions. If several options have a high conformity, then they may all be adequate treatments and the patient may have a choice.

If all options have low conformity, then there may be no common approach applied in tumor boards, for example, when many novel, experimental therapies are emerging but no clear guidelines are available, or the specific condition combination of this patient is rare and each case has to be decided individually. Here the patient may intentionally select a very novel therapy with high risk.

The approach is based on comparing a given patient to a patient population retrospectively after the treatment decision (which provides the recommended treatment). In this way, it is possible to provide a simple implementation based on currently documented data only and to provide both patients and clinicians with valuable information of the relationship to a suitable reference group. This is a clear advantage over the many data-mining based proposals, which require access to all kinds of currently non-documented data. A further advantage is that the approach does not judge the treatment recommendation offensively as ‘good’ or ‘bad’, but simply as ‘common’ or ‘not common’.

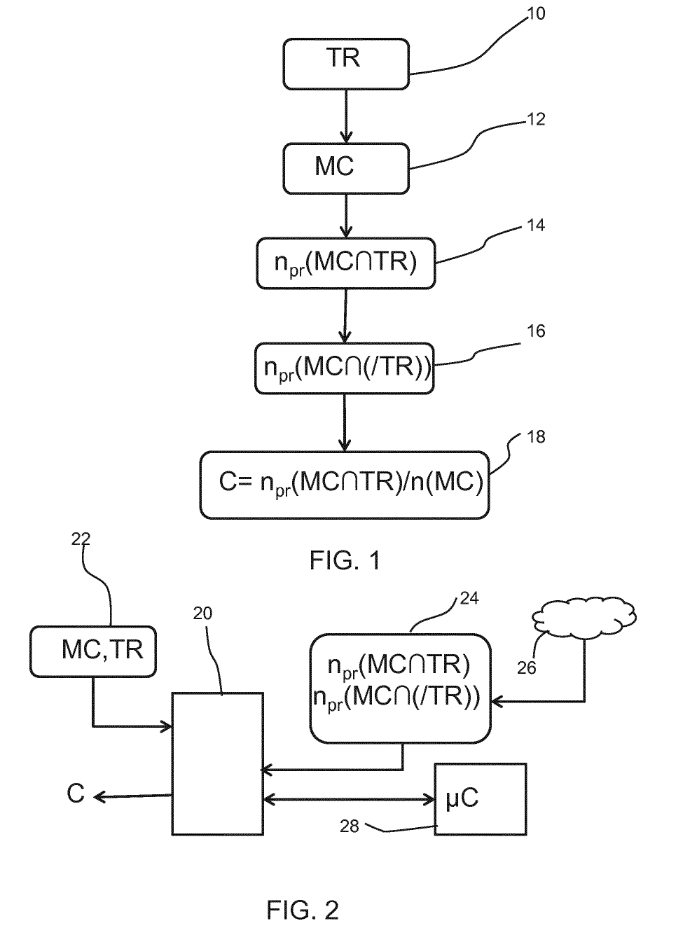

FIG. 1 shows a method of generating an assessment of a treatment recommendation for a patient. It is implemented by a computer based system.

In step 10, a treatment recommendation (TR) for the patient is provided to (and received by) the system. This treatment recommendation is for example reached at by a particular board of specialists associated with one particular hospital.

In step 12, information is provided to (and received by) the system about the medical condition (MC) of the patient.

This includes at least the patient diagnosis, such as their type and stage of cancer. However, it may include additional patient-specific information such as (one or more of) the patient's physical condition (weight, blood pressure, heart rate, etc. and/or more specific details about the medical condition), the patient family history (in respect of the medical condition) and biomarker information for the patient. The medical condition may be defined as a combination of these parameters, such that there is a sufficient number of similar patients for data analysis, but the similar patients have sufficiently similar medical-related parameters that they can be expected to react in a similar way to different treatments.

In step 14, information is provided to (and received by) the system relating to other patients. This information includes the number of previous patients (nₚᵣ) who have received the recommended treatment (TR) and had a medical condition (MC) corresponding to that of the patient. The medical condition may be the same in that it relates to the same type of cancer for example, or it may the same in even more detail, for example the same type of cancer and the same tumor size, or specific tumor location or degree of spreading. The information about previous patients includes patients for whom the treatment recommendation was made by a different panel of specialists, and furthermore associated with different medical institutions. Thus, the number of previous patients includes patients treated by a plurality of different treatment specialists.

This historical information (of previous patient treatment information and medical conditions) may be derived from health insurance information. This is for example available by accessing database information over the internet. The historical information may additionally be extracted from electronic medical records associated with the patients for whom treatment has been given.

In step 16. information is provided to (and received by) the system including the number of previous patients who had a medical condition corresponding to that of the patient but received one or more alternative treatment options (/TR).

In step 18, a measure of conformity of the treatment recommendation to the medical condition of the patient is provided. It is based on the treatments received by previous patients having a medical condition corresponding to that of the patient.

The measure of conformity indicates how usual or unusual the recommended treatment is, having regard to the medical information. It is a statistical measure which is of interest for the patient and for medical professionals to judge how typical the recommendation is.

The measure of conformity for example comprises a ratio of (i) the number of previous patients with matching information about their medical condition who had the treatment corresponding to the treatment recommendation (i.e. the number of previous patients having corresponding MC and TR), to (ii) the total number of patients with matching information about their medical condition (i.e. the number of previous patients having corresponding MC but including all possible treatments (TR and/TR)).

There may be multiple measures of conformity, for example relating to different types of patient-specific information. For example, conformity of the recommended treatment in respect of patients with the same biomarker information may be of interest,

FIG. 2 shows a system for generating an assessment of a treatment recommendation for a patient.

The system comprises an input/output interface 20, which receives as input the medical condition and recommended treatment (shown as block 22) and the historical information about previous patients with the same medical condition (shown as block 24). Some or all of this information may be extracted from the internet 26 (for example from insurance databases), and some or all may be input manually to the input/output interface 20.

The input/output interface 20 provides the conformity measure (C) as output.

The data processing is carried out by a processor 28, which runs suitable software.

An example of how the method may be used will now be explained.

It is assumed that a male patient has prostate cancer. He has already consulted three tumor boards, which decided for three treatment options T1 (chemotherapy), T2 (radiation therapy) and T3 (surgery). The patient decides for treatment T1 (chemotherapy).

The treatment procedures in the hospital will generate specific costs which are known in advance. Hospitals usually encode their costs on patient basis of a DRG (diagnosis related group) type system. The data (patient ID, diagnosis, procedures) is sent to the health insurance companies for reimbursement purposes. There is for example a central organization for cost reimbursement in hospitals.

The relevant health authority provides statistical information for the different hospitals each year, based on these DRG codes, partly available to the public. Therefore, for each diagnosis the procedures which have been applied are known, and also the patients (their IDs) are known. Thus, there are three groups of patients (all with the same diagnosis, i.e. medical condition) P1, P2 and P3, associated with the three treatment options T1, T2, T3. For each treatment option T1, T2 and T3 the size of the group of patients is known (based on their IDs). The patients in the group will have been treated in many different hospitals and therefore their diagnoses will have been given by different medical specialists.

Each patient in a group P1, P2, P3 has an electronic medical record (EMR). The EMR provides disease specific information, such as patient condition, family history and presence of biomarkers. In prostate cancer for example, the level of a certain protein in the blood (PSA) is used for risk stratification and therapy selection. The PSA value is measured in nanograms per milliliter, high values may recommend further procedures (imaging) and rule out certain treatment options.

Based on these PSA values, a histogram can be derived where the patient groups are indicated.

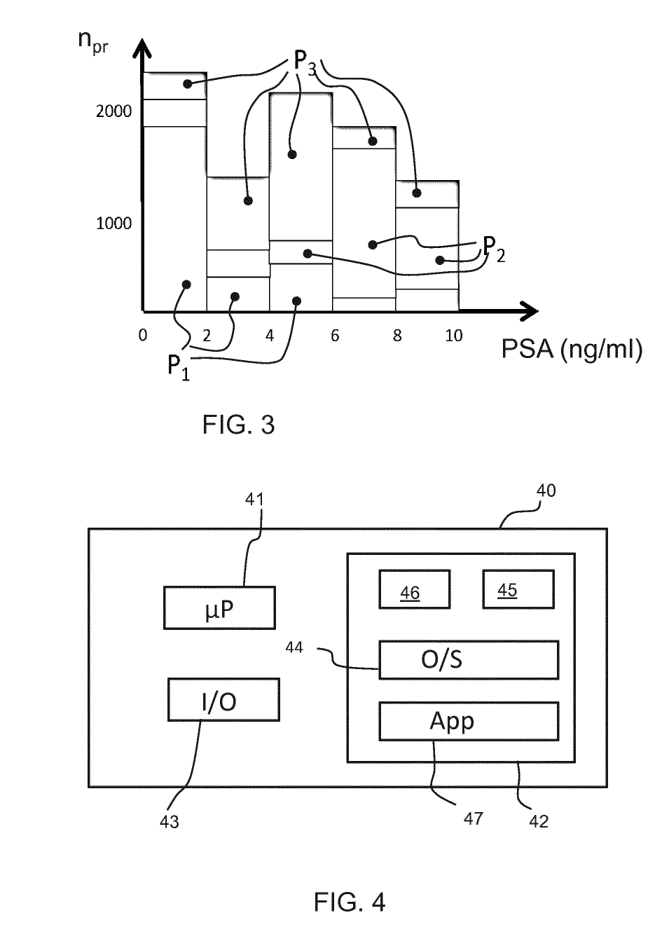

FIG. 3 shows a histogram of patients' PSA values where the patient groups P1 (receiving chemotherapy), P2 (receiving radiotherapy) and P3 (receiving surgery) are shown. In this example, for low PSA values chemotherapy (i.e. patients in group P1) is most common, for intermediate PSA values surgery (i.e. patients in group P3) is most common and for high PSA values radiotherapy (i.e. patients in group P2) is the most common treatment.

For example, a patient receiving therapy option T1 (chemotherapy) has a PSA value of 0.5 ng/ml. In the histogram in FIG. 3 this patient is represented in the left most bar in patient group P1. In this bar almost all patients receive chemotherapy, only relatively few patients are in the groups P2 and P3 (radiation therapy and surgery, respectively). Hence the conformity of the therapy T1 for the patient is high.

If the patient has a higher PSA of 6.5 ng/ml (histogram bar number 4), many more patients are from group P2 (who received radiation therapy) and chemotherapy has a lower conformity for the specific PSA value.

The conformity of a therapy option may be defined as the number of patients of the same group (i.e. having had the same treatment as has been recommended for the patient), divided by the total number of patients for that specific histogram bar (i.e. for those patients with the same medical condition but including all treatment options). In this case, the same medical condition is sufficiently narrow to include only a particular range of PSA values.

The conformity is then in the range of [0, 1]. The higher the value, the better is the conformity of the treatment with respect to the chosen biomarker.

Other parameters in the electronic medical records can be evaluated with the same statistical analysis providing further insights into the treatment conformity or non-conformity. In a similar way also more than one parameter can be investigated by using higher dimensional histograms.

The patient may then choose the treatment option with best conformity in the patient group. The patient may also use this information to discuss with his oncologist, why the oncologist recommended a treatment even though the patient does not seem to have the same conditions of other patients receiving this treatment. The tumor boards themselves may also evaluate their decisions on a regular basis in the same way, for example, to question non-conforming treatment options and then document reasons for diverging.

This method and system can be applied to any treatment in oncology, not only to the specific example of prostate cancer. It may also be applied to other medical conditions where there are multiple treatment options.

The algorithm is not based on information drawn from guidelines nor from databases providing information on the outcome of therapies, but is based on treatments which have been given.

Other conformity indices can be calculated by subdivision of the patient database, for example conformity scores may be region-based comparing different countries or individual hospitals of a hospital chain.

The conformity measure described above is based on finding the number of patients with same medical properties and the same treatment, relative to the number of patients with same treatment. Other variations are possible.

If the patients with the same treatment and a diverse range of available medical condition properties fall into very small groups, some of the medical condition properties may be ignored to obtain larger groups and a more informative conformity index.

Thus, the number of parameters which define the medical condition of the patient may be made variable so that desired group sizes may be obtained.

The total number of patients who have received a particular treatment may be made available, because the conformity index is of course of limited value if only very few patients receive this treatment. Further, the time-span could be controllable. For example, a new treatment might become the gold-standard, but most patients still received the previous gold-standard.

The conformity measure may be presented to different users, for example:

(i) at tumor board (or multidisciplinary team) meetings to encourage extended documentation of non-conform recommendations;
(ii) at patient consultation, to explain the treatment recommendation to the patient in relation to standard care;
(iii) at guideline decision meetings of experts to revise or define new guidelines or standards of care;
(iv) for insurance companies to evaluate the properness of a subscribed treatment.

Individual patients may be clustered into groups, for example groups based on hospital, hospital chain, county or country. The conformity measure may then be determined in each cluster separately. Comparing the conformity measures between the clusters may also give interesting insights on conformity.

The conformity measure may be used as a hospital-specific key performance indicator (patient-wise, or hospital-wise within a hospital chain) which is displayed as part of a dashboard or administration benchmarking tool. Hence the conformity measure may be added to other software tools and processes.

The system described above makes use of a controller for processing data.

FIG. 4 illustrates an example of a computer 40 for implementing the controller described above.

The computer 40 includes, but is not limited to, PCs, workstations, laptops, PDAs, palm devices, servers, storages, and the like. Generally, in terms of hardware architecture, the computer 40 may include one or more processors 41, memory 42, and one or more I/O devices 43 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 41 is a hardware device for executing software that can be stored in the memory 42. The processor 41 can be virtually any custom made or commercially available processor, a central processing unit (CPU), a digital signal processor (DSP), or an auxiliary processor among several processors associated with the computer 40, and the processor 41 may be a semiconductor based microprocessor (in the form of a microchip).

The memory 42 can include any one or combination of volatile memory elements (e.g., random access memory (RAM), such as dynamic random access memory (DRAM), static random access memory (SRAM), etc.) and non-volatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 42 may incorporate electronic, magnetic, optical, and/or other types of storage media. Note that the memory 42 can have a distributed architecture, where various components are situated remote from one another, but can be accessed by the processor 41.

The software in the memory 42 may include one or more separate programs, each of which comprises an ordered listing of executable instructions for implementing logical functions. The software in the memory 42 includes a suitable operating system (O/S) 44, compiler 45, source code 46, and one or more applications 47 in accordance with exemplary embodiments.

The application 47 comprises numerous functional components such as computational units, logic, functional units, processes, operations, virtual entities, and/or modules.

The operating system 44 controls the execution of computer programs, and provides scheduling, input-output control, file and data management, memory management, and communication control and related services.

Application 47 may be a source program, executable program (object code), script, or any other entity comprising a set of instructions to be performed. When a source program, then the program is usually translated via a compiler (such as the compiler 45), assembler, interpreter, or the like, which may or may not be included within the memory 42, so as to operate properly in connection with the operating system 44. Furthermore, the application 47 can be written as an object oriented programming language, which has classes of data and methods, or a procedure programming language, which has routines, subroutines, and/or functions, for example but not limited to, C, C++, C#, Pascal, BASIC, API calls, HTML, XHTML, XML, ASP scripts, JavaScript, FORTRAN, COBOL, Perl, Java, ADA, .NET, and the like.

The I/O devices 43 may include input devices such as, for example but not limited to, a mouse, keyboard, scanner, microphone, camera, etc. Furthermore, the I/O devices 43 may also include output devices, for example but not limited to a printer, display, etc. Finally, the I/O devices 43 may further include devices that communicate both inputs and outputs, for instance but not limited to, a network interface controller (NIC) or modulator/demodulator (for accessing remote devices, other files, devices, systems, or a network), a radio frequency (RF) or other transceiver, a telephonic interface, a bridge, a router, etc. The I/O devices 43 also include components for communicating over various networks, such as the Internet or intranet.

When the computer 40 is in operation, the processor 41 is configured to execute software stored within the memory 42, to communicate data to and from the memory 42, and to generally control operations of the computer 40 pursuant to the software. The application 47 and the operating system 44 are read, in whole or in part, by the processor 41, perhaps buffered within the processor 41, and then executed.

When the application 47 is implemented in software it should be noted that the application 47 can be stored on virtually any computer readable medium for use by or in connection with any computer related system or method. In the context of this document, a computer readable medium may be an electronic, magnetic, optical, or other physical device or means that can contain or store a computer program for use by or in connection with a computer related system or method.

The invention is of interest as part of a patient portal in oncology, or for medical professionals, in each case to support diagnosis and decision making for cancer patients. It may be used by tumor boards for tracking conformity. It may also be used as part of a system which interfaces with a medical procedure billing-system.

The conformity measure does not present a suggestion to clinicians, but only relates a clinical decision (treatment recommendation) to the available data. It remains the clinicians' responsibility to interpret this information and potentially react.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A method of generating an assessment of a treatment recommendation for a patient, comprising:
receiving a treatment recommendation for the patient;
receiving information about the medical condition of the patient;
receiving information including the number of previous patients who have received the recommended treatment and had a medical condition corresponding to that of the patient, wherein the number of previous patients include patients treated by a plurality of different treatment specialists;
receiving information including the number of previous patients who had a medical condition corresponding to that of the patient but received one or more alternative treatment options; and
providing a measure of conformity of the treatment recommendation to the medical condition of the patient based on the treatments received by previous patients having a medical condition corresponding to that of the patient;
wherein the method comprises consulting health insurance information to obtain the previous patient treatment information and medical conditions.

2. A method as claimed in claim 1, wherein the measure of conformity comprises the ratio of:
(i) the number of previous patients with matching information about their medical condition who had the treatment corresponding to the treatment recommendation, to
(ii) the total number of patients with matching information about their medical condition.

3. A method as claimed in claim 1, wherein the information about the medical condition of the patient further comprises patient-specific information relating to the medical condition.

4. A method as claimed in claim 3, wherein the patient-specific information comprises one or more of:
patient condition;
patient family history; and
biomarker information for the patient.

5. A method as claimed in claim 3, comprising generating a plurality of measures of conformity relating to different types of patient-specific information.

6. A method as claimed in claim 5, comprising, for one or more of the different types of patient-specific information, generating a histogram which records the different treatments received by different previous patients for different values of the particular type of patient-specific information.

7. A method as claimed in claim 1, wherein the medical condition comprises a cancerous tumor.

8. A method as claimed in claim 1, wherein the treatment recommendation comprises chemotherapy, surgery or radiotherapy.

9. A computer program comprising code means which is adapted, when said program is run on a computer, to implement the method of claim 1.

10. A system for generating an assessment of a treatment recommendation for a patient, comprising:
an input for receiving:
a treatment recommendation for the patient;
information about the medical condition of the patient;
including the number of previous patients who have received the recommended treatment and had a medical condition corresponding to that of the patient, wherein the number of previous patients include patients treated by a plurality of different treatment specialists; and
information including the number of previous patients who had a medical condition corresponding to that of the patient but received one or more alternative treatment options; and
a processor, which is adapted to:
provide a measure of conformity of the treatment recommendation to the medical condition of the patient based on the treatments received by previous patients having a medical condition corresponding to that of the patient; and
consult health insurance information to obtain the previous patient treatment information and medical conditions.

11. A system as claimed in claim 10, wherein the processor is adapted to determine, as the measure of conformity, the ratio of:
(i) the number of previous patients with matching information about their medical condition who had the treatment corresponding to the treatment recommendation, to
(ii) the total number of patients with matching information about their medical condition.

12. A system as claimed in claim 10, wherein the information about the medical condition of the patient further comprises patient-specific information relating to the medical condition, wherein the patient-specific information comprises one or more of:
patient condition;
patient family history; and
biomarker information for the patient.

13. A system as claimed in claim 12, wherein the processor is adapted to:
generate a plurality of measures of conformity relating to different types of patient-specific information.
